SEQUENCE LISTING (4 sequences)

SEQ ID NO: 1 - protein, 1073 aa, Nezara viridula totivirus
MEESQTPIAGPLSSSKKVIPSTTQLVGLASWIAKPLDIVGPKTSNLYSAPSARDSVSRAGCGSHFFTSWESYSRSYFGGLSFLTDQEVSRTRRSVQLPDQTGDTPTPEEMSQMNRTEKMILSKGGIFLRKRLPGIITTSIIMTPTQIGLTKNRRLINIASKYDQYVREGLTPEQAANKIELERQQPAPQSTQSQQPQDPTRTETLSGDESVAGGPSDPTPSGSRTKRQVTARLDVISNTIPVRCVQSTSWKTDEQLSQSIPYSAIYTSIVQRLNDPSATGALGALARGPSIRDGTSLISLAAHLANDTGSVMEIAPVVRATCMGLSVSSECYDTDPIQFRVFRNFTRTEVTATHIHKPDVPPVTCNIMAVGVDIFTSFMIDKTGPLDQAGDFKYQNADVTWTAVPVRAAMMNQFYIIPYIASFVTSELWAGRTSFNIGGNYTLSTGTNVSAEINLLPCANNVYIPGPADILLVLLDSSSTFAQTNLTIMGTQVPLYGGAPVNPVPFTTMFDDWFKEANVGRIPMDCATAVNTISSMLGVENSTNLGLSIAAELYGCQFNGINIPVIASTIGPPTPDYTKPGGGAYRIDLQDVSMQTGSPIIAKGITTNAALITPLITSYNFTGISPWCLPPTGMVKTKSVTVGTDTYPVWAQAVPDYYSFNYTATTVNSVARVAIAMQLVITTELSIKFNYGGGFSTWMHMLSGALCAQASTIFSSTNINLSIWTGYNITEDEVFSTQYMMEAIKIATNGIANFQKYQQLVNTWTGFDPALISKYFGLPVFEDPNWLSYHPLPYYYAVQWLQKHKSVEGTAPAHVVNWYHDKLSRMGLLIDETTRDYRPFITGSPNIQTYCPIVTAETIQEPELKHMDMWIDEFSYITNISAKNTSTGKPRLLTSQFFVLSPVDSGLAYTPGLKIYVYNSAYENFSPERRDAVFSPLMYPDPPTLREIVEGAKHWLLYPGLSALAGFVTGGPAGAAIAGGSHLAKNVLDRIVSDPDQKQQISEAVQKVARQAVAPAPPETITSPVQALTQSILNPEKYPTIPIQTQTPQVPPSVSTTVEHPVNPLSTLVTPTT

SEQ ID NO: 2 - protein, 647 aa, Helicoverpa armigera stunt virus
MGDAGVASQRPHNRRGTRNVRVSANTVTVNGRRNQRRRTGRQVSPPDNFTAAAQDLAQSLDANTVTFPANISSMPEFRNWAKGKIDLDSDSIGWYFKYLDPAGATESARAVGEYSKIPDGLVKFSVDAEIREIYNEECPVVTDVSVPLDGRQWSLSIFSFPMFRTAYVAVANVENKEMSLDVVNDLIEWLNNLADWRYVVDSEQWINFTNDTTYYVRIRVLRPTYDVPDPTEGLVRTVSDYRLTYKAITCEANMPTLVDQGFWIGGQYALTPTSLPQYDVSEAYALHTLTFARPSSAAALAFVWAGLPQGGTAPAGTPAWEQASSGGYLTWRHNGTTFPAGSVSYVLPEGFALERYDPNDGSWTDFASAGDTVTFRQVAVDEVVVTNNPAGGGSAPTFTVRVPPSNAYTNTVFRNTLLETRPSSRRLELPMPPADFGQTVANNPKIEQSLLKETLGCYLVHSKMRNPVFQLTPASSFGAVSFNNPGYERTRDLPDYTGIRDSFDQNMSTAVAHFRSLSHSCSIVTKTYQGWEGVTNVNTPFGQFAHAGLLKNEEILCLADDLATRLTGVYPATDNFAAAVSAFAANMLSSVLKSEATSSIIKSVGETAVGAAQSGLAKLPGLLMSVPGKIAARVRARRARRRAARAN

SEQ ID NO: 3 - protein, 237 aa, Tymoviridae sp. virus
MEEIVPLVASAASAIPSLVNAFSSSKTTPSDSSPSRPLDMQPLPSTDSVVLSSQSLPPAPPPPLGASVGRSSSRLIIPFQRLYFDLTGTETKSNSVTVQSLPNVASIIKGYRDAYLVDLEAVVFPSAPSLKIPVTVDLCWTTADVTVEGTNVLATPSSARITIGGLALMHQATLPCDLGYINPIIKSPIPYTNHPRLNIHFHQNPDAVSEGTSTKTKASVVIRGSISVSHPLVTGHS

SEQ ID NO: 4 - protein, 610 aa, Spodoptera frugiperda rhabdovirus
MVFLSLSTIIFILSLRAVTCSNPLSYPNGILTNNSTHNHPLSDFYIFYENSSLTYTQFPVAPDCSSILDTRDEQYPTTVTLWKVDQESQAEWGLLLWQERIDTTCSWNFWGNYKGSIVSKSSVPLKDIPSGSARNGYWALSNDEVQEIDHVPYNLRYYCYWCRNEYPGSFYMRYVKKVRIIRNPDGSIKTPRGSWVHELDNLWGDQMRYLVIRRFGGESSCPLKIYDVRAGVLSKSRSNFILVSLPSLNLQFSVSLESTETKCSFGDKTYDIVQSMGGYLLSIDIGNANWRGPWDPTPQHPGRERRSIMEFPDQTSFRYNQFINYHSSPRHKRHDQEFEFPLSLKSSYDYAQFRYEQNFIIRQINKNFGLLQKSICDIQFSKWQNLSPPNLAMKIAHYVTGSIHSIGGVHHGSYSIQRTEKSITKVNLVFPIVIVHGMYKCQREPSKEVVWAEPVTGILFKSPIPTHFSLSSSWLPGVNGSSIVPLTGQILLPEITMDHLEVVQQVEAKMVKSMYTNVELFGSTEEFQRYQTQGITSDEQSNTVNPWIGLLIHGGVSIATGILVALLIPSILKLFRHIIEKGEASLEERLHLRETSRKEFVKVRGKPWGV

(19) United States
(12) Patent Application Publication
(10) Pub. No.: US 2024/0360468 A1
(43) Pub. Date: Oct. 31, 2024

(54) VIRAL COAT DELIVERY OF INSECT RESISTANCE GENES IN PLANTS

(71) Applicant: BASF Agricultural Solutions Seed US LLC, Florham Park, NJ (US)

(72) Inventors: Kavita Bitra, Research Triangle Park, NC (US); Alberto Bressan, Research Triangle Park, NC (US)

(73) Assignee: BASF Agricultural Solutions Seed US LLC, Florham Park, NJ (US)

(21) Appl. No.: 18/291,282

(22) PCT Filed: Jul. 26, 2022
(86) PCT No.: PCT/US2022/074158

§ 371 Date: Jan. 23, 2024

Related U.S. Application Data
(60) Provisional application No. 63/225,975, filed on Jul. 27, 2021.

(51) Int. Cl. C12N 15/82; C07K 14/005

(52) U.S. Cl. CPC C12N 15/8286; C07K 14/005; C12N 2720/00022; C12N 2760/20022; C12N 2770/00022; C12N 2770/40022

(57) ABSTRACT

Herein, is describe a new technology that exploits modified-virus-like particles (VLP) to improve deliverability and increase effectiveness of dsRNAs/chemicals/peptides to difficult-to-control phytophagous insect pests, including hemipteran and lepidopteran insects. This technology has also the potential to control plant pathogens including fungi, like Asian Soybean Rust (ASR).

### RELATED APPLICATIONS

This application claims the benefit of U.S. Provisional Application Ser. No. 63/225,975 filed Jul. 27, 2021, the contents of which are herein incorporated by reference in its entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 202609_SEQLISTING_Std26.xml. The size of the xml file is 7 KB, and the file was created on Jul. 25, 2022.

### FIELD OF THE INVENTION

This invention relates to the field of molecular biology. Provided are novel modified-virus-like particles (VLPs) for in-vitro and in-planta production to improve deliverability and increase effectiveness of dsRNAs, chemicals compounds, or peptides to difficult-to-control phytophagous insect pests, plant fungal pathogens or to increase yield and/or agronomic performance in a plant.

### BACKGROUND OF THE INVENTION

Insects are infected by several different viruses. Some are maintained within insects as sole hosts and can be pathogenic, others like plant viruses exploit insects as vectors.

Both insect viruses and insect-vectored plant viruses have the ability to enter into the insect body through the insect feeding process and gain access to the gut tissue. In this process viral particles are ingested with food and withstand insect digestive enzymes in the gut lumen. The viral particles are next internalized into the gut tissue through specific cellular process (i.e. receptor mediated endocytosis/lipid raft/caveolae etc.). In several cases, viral internalization is mediated by the viral coat proteins.

It has been noted that viral coat proteins seem to: 1. mediate specificity to certain insect species; 2. provide resistance of the viral genetic material to the insect gut digestive system; and 3. mediate entry into the insect gut cells through different cellular transport processes. This invention intends to exploit these viral proteins, and the genes encoding them, for presentation and delivery of insect resistance genes, gene silencing molecules or other proteins of interest to target pests or other organisms. Furthermore, the viral proteins can be genetically modified to interact with a wider range of organisms than occurs in nature. By facilitating efficient delivery of insect resistance genes, this invention can be used to genetically engineer pest-resistant plants, or to construct biopesticides that can be directly applied via spray-on application.

In one embodiment of the invention, any virus which can infect the insect gut and enter the hemocoel of an insect can be employed, as described, to deliver a toxin or cargo molecules to the insect. Herein, “cargo molecule” or “cargo” are interchangeably used to indicate a protein, DNA/RNA molecule or chemical that will be encapsulated in the viral-like particles for example a cargo molecule can be an enzyme, an insect toxin such as a Cry1 gene, or dsRNA that are active against insect or fungal pathogens.

One approach toward insect pest control has been the use of baculoviruses, which are insect pathogenic viruses. Some of these viruses have been used to deliver a variety of insect-specific toxins that are active in the hemocoel but not in the gut of the insect. For example, recombinant baculoviruses have been developed for control of lepidopteran (moth) pest species (Bonning, B. C. et al. [1996] Annu. Rev. Entomol. 41:191-210). These baculoviruses have been engineered to produce insect hormones such as diuretic hormone (Maeda, S. [1989] Biochem. Biophys. Res. Comm. 165:1177-1183), enzymes such as juvenile hormone esterase (Bonning, B. C. et al. [1997] Proc. Natl. Acad. Sci. USA 94:6007-6012), and insect-specific toxins derived from venomous species such as scorpions and parasitic wasps (McCutchen, B. F. and Hammock, B. D. [1994] in Natural and Derived Pest Management Agents, Hedin, P. et al. [eds.], #551 ed., Washington, D.C.: Am. Chem. Soc., pp. 348-367. ACS Symposium Series; Hughes, P. R. et al. [1997] J. Invert. Pathol. 69:112-118; Lu, A. et al. [1996] in Biological control: theory and applications in 1996. 7:320; Gershburg, E. et al. [1998] FEBS Lett. 422:132-136; Jarvis, D. L. et al. [1996] in Biological control: theory and applications in 1996, 7:228). Bonning et. al also showed that insect resistance genes were successfully delivered to various insect pests (see U.S. Pat. No. 7,312,080 herein incorporated in its entirety by reference).

Several viral capsid proteins have the ability to self-assemble in vitro or when expressed in a plant cell, creating viral-like particles, (herein, “VLPs”). VLPs allow for creating complexes with cargo molecules thereby promoting delivery of cargos into the gut tissues of target pests. The use of viruses that are specific to target pests allow to increase efficiency and specificity to the intended target pest. What is needed are novel VLPs that can be linked to gene silencing molecules, insecticidal peptides or chemicals specifically targeted to control commercial insect pests and/or pathogens.

### SUMMARY OF INVENTION

Certain aspects of this invention involves combining a nucleotide encoding a peptide toxin or dsRNAs for gene silencing or small molecules (i.e. Cargo Molecules) with insecticidal activity, with a transport peptide derived from insect-specific VLPs capable of complexing with cargos and facilitating internalization into the insect gut cells. The combination can be achieved by a fusion of genetic material encoding the peptide toxin and the VLPs (herein, “Fusion VLPs”), such that expression of the Fusion VLPs that results in synthesis of a fusion protein combining the functions of both the insecticidal molecule and the viral transport peptide.

Alternatively, this technology allows for the complexing of the viral coat proteins with cargos due to viral coat protein's ability to self-assemble into VLPs and recruit active molecules, resulting in a encapsulation of the Cargo Molecule (herein, “encapsulated VLP”). Ingestion of the fusion protein or encapsulated VLPs by the insect allow internalization into the gut tissue improving the efficacy of cargo molecules and their specificity that can now access the intracellular tissues of target pests. In one embodiment of the invention the fusion protein or the encapsulated VLPs is effective against a plant pest or pathogen. In a preferred embodiment, the invention is effective in control of a Lepidopteran or Hemipteran insect pest. A variety of insect-specific viral coat proteins can be used as transporter peptides/VLPs creating a high target-pest specific solution. It is also envisioned that the encapsulated VLP could also be used to deliver other useful genes, nucleotide sequences, chemicals or genetic elements into the plant cell that could confer benefits such as disease resistance, increased yield, compounds impacting plant growth or development, or other agronomic factors.

Any peptide having an insecticidal effect when present in a target insect can be incorporated into either a fusion VLP or encapsulated VLP as taught herein. Virus proteins that cross the gut barrier of an insect or other pest organism can be exploited for direct delivery of a variety of toxic agents which are active only in the body cavity of that organism and that would encounter degradation in the gut lumen if exposed directly to the enzymatic activity of the insect digestive system. Methods described herein, overcome this issue by either forming fusion VLPs or encapsulating VLPs. The requirements for these toxic agents include that (1) the agent should be specific for the targeted pest without mammalian toxicity; (2) the agent should be active at low levels; (3) the agent should have a rapid effect on the host. These toxic agents include both toxins that act on the nervous system of insects, and physiological effectors which disrupt regulation of homeostasis in the insect, resulting in feeding inhibition and/or death. A variety of such toxins and physiological effectors have already been exploited specifically for the control of lepidopteran (moth) pests by recombinant baculovirus expression. The insect-specific neurotoxins have generally been considered to be more effective than physiological effectors, in part because of feed-back regulatory systems in the insect for the latter. There is an ongoing effort within commercial, government and academic laboratories to isolate toxins that specifically target the insect nervous system from a variety of organisms that use venoms to immobilize their prey. The virus coat protein delivery system (Fusion VLPs or Encapsulated VLPs) can be exploited for delivery of all of these agents in an array of pest species.

The compositions and methods of the invention are useful for the production of plants with enhanced pest resistance or tolerance (e.g. against insects, fungal pathogens, etc.). These organisms and compositions comprising the organisms are desirable for agricultural purposes. The compositions of the invention are also useful for generating altered or improved proteins that have pesticidal activity, or for detecting the presence of pesticidal proteins or nucleic acids in products or organisms.

### BRIEF DESCRIPTION OF THE DRAWINGS

FIG. 1: SDS-PAGE of virus preparation from Nezara viridula.

FIG. 2: Schematic example of plant genetic engineering approach for creating expression constructs utilizing the viral coat protein and cargo molecule.

FIG. 3: HaSV SDS-PAGE and Western Blot analysis.

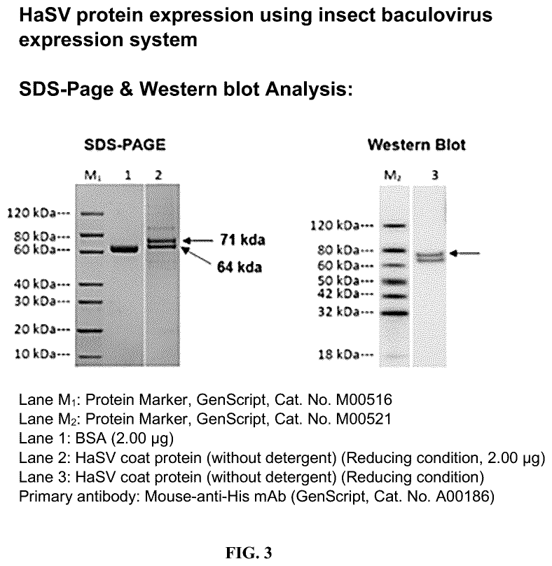

FIG. 4: Relative amounts of encapsulated eGFP following post-enzymatic treatment.

### DETAILED DESCRIPTION

Aspects of the invention allows the person having ordinary skill in the art to construct either fusion VLPs or encapsulated VLPs by operably fusing a nucleotide encoding a transport peptide (e.g. any of SEQ ID NO:s 1-4) and an insect-toxic peptide for control of insects (e.g. Lepidopteran or Hemipteran pest) into a plant expression cassette. Such plants include, but are not limited to, wheat, barley, oats, rice, corn, oil seed rapeseed such as canola, potato, sugar beet, soybean, tomato, citrus (orange, lemon, lime, grapefruit), Rosaceae (rose), fruit trees (plum, apple, cherry, peach, pear), lettuce, french bean, sugar cane, papaya, squash, cucurbits, banana, cassava, sweet potato, grape, all ornamentals and the like, including other members of the plant families to which the foregoing plants belong.

In one embodiment, the invention provides for delivery of traits for pest control (herein, “insect traits”). Insect viral coats from Helicoverpa armigera stunt virus (HaSV), e.g. SEQ ID NO: 2)), Spodoptera frugiperda macula like virus (SfMLV), e.g. SEQ ID NO: 3)), Spodoptera frugiperda rhandovirus G protein (SfG protein), e.g. SEQ ID NO: 4)) and Nezara viridula Totivirus (NvTV), SEQ ID NO: 1)) can be modified to generate virus like particles (VLPs) and packaged for delivery of insect resistance peptides, or other insect traits to respective insect pests. All the listed insect viral coats (see SEQ ID Nos 1-4) can be fused with an insect trait to deliver traits as monomeric forms. Insect traits can be expressed as a protein, peptide, double strand RNA (dsRNA) and/or a small molecule. Various aspects of the invention can be used as a transgenic approach or as a spray-on formulation.

The fusion VLPs or encapsulated VLPs can be delivered to the target insect by any of a variety of ways (e.g. spray application, etc.). However, a preferred method is to deliver the fusion VLPs or encapsulated VLPs during the natural feeding activity of the insect, from the plant itself through a transgenic approach. The invention therefore includes plant-expressible gene constructs which can be used to transform a plant such that the plant expresses the fusion VLPs or encapsulated VLPs in its sap or tissues where insect feeding occurs. Transgenic plants, capable of expressing a fusion VLPs or encapsulated VLPs protein are thereby rendered resistant to the damage caused by insects and the diseases transmitted by them. The plant expressible gene constructs encoding for fusion VLPs or encapsulated VLPs can be expressed either constitutively, or in an inducible manner, such as during a desired developmental stage, in a desired tissue, at a desired time or under desired environmental conditions like insect attack. Constructs can be expressed from stably integrated transgenes or via transient vectors. Modified fusion VLPs or encapsulated VLPs can also be used as a spray on the surface of the crop plant, where ingestion by the target insect introduces the toxic protein or other insect traits to the insect gut.

The term “peptide” is used to refer to any poly-amino acid, without limitation as to size or molecular weight. As used herein, “peptide” includes such terms of common usage in the art as “oligo-peptide,” “polypeptide” and “protein.”

The term “transport peptide” is herein defined as that peptide segment which is necessary for transport of a circulatively-transmitted virus from the gut to the hemocoel of an insect. A transport peptide can include all, or a portion of, a virus coat protein or other virus protein and can also include all or part of a readthrough domain. That portion of a coat protein or other virus protein which constitutes a transport peptide is termed a component of the coat or other protein. It will be understood in the art that a specific interaction exists between the transport peptide of a virus and the insect host of the virus. A peptide intended to serve as a transport peptide for a given insect species is obtained from a virus that is known to infect that insect, as would be understood in the art. One aspect of the invention involves an operable fusion of a nucleotide encoding an insect-toxic peptide to a nucleotide encoding any one of the transport peptides identified in SEQ ID Nos: 1-4 all operably linked to a plant promoter which when expressed in a plant form a fusion peptide that can control an insect.

The term “insect-toxic peptide” or “pesticidal protein” refers to any peptide which is toxic to an insect when delivered to the appropriate site of action of the insect. The present invention is directed to toxic peptides which exert their effect when delivered to the hemocoel of the insect. Examples of insect-toxic peptides are well known in the art. In one aspect of the invention a nucleotide sequences encoding transport peptides identified in SEQ ID Nos: 1-4 are operably fused to a nucleotide sequence encoding an insect-toxic peptide active against either Lepidopteran or Hemipteran pest (e.g. MTX class of genes or Axmi486). “Pesticidal gene” means a nucleotide sequence that encodes a pesticidal protein or insect-toxic peptide.

The term “fusion peptide” is herein defined as the operable fusion of a transport protein according to the invention with a binding protein for RNA, dsRNA or for other small molecules with pesticidal activity

A segment of coding DNA is “expressed” in vivo or in vitro, if the DNA is transcribed or if the transcription product is translated. Expression can result in synthesis of an mRNA or of a protein encoded by the coding DNA.

“Associated with/operatively linked” refer to nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric gene” is a recombinant nucleic acid sequence in which a promoter or regulatory nucleic acid sequence is operatively linked to, or associated with, a nucleic acid sequence that codes for an mRNA or which is expressed as a protein, such that the regulator nucleic acid sequence is able to regulate transcription or expression of the associated nucleic acid sequence. The regulator nucleic acid sequence of the chimeric gene is not normally operatively linked to the associated nucleic acid sequence as found in nature. A chimeric gene having operatively linked coding and expression control segments is also referred to herein as an “expression cassette.”

To “control” insects means to inhibit, through a toxic effect, the ability of insect pests to survive, grow, feed, and/or reproduce, or to limit insect-related damage or loss in crop plants. to “control” insects may or may not mean killing the insects, although it preferably means killing the insects.

To “deliver” a toxin means that the toxin comes in contact with an insect, resulting in toxic effect and control of the insect. The toxin can be delivered in many recognized ways, e.g., orally by ingestion by the insect or by contact with the insect via transgenic plant expression, formulated protein compositions(s), sprayable protein composition(s), a bait matrix, or any other art-recognized toxin delivery system.

A “plant” is any plant at any stage of development, particularly a seed plant.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant tissue” as used herein means a group of plant cells organized into a structural and functional unit. Any tissue of a plant in plants or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A “promoter” is an untranslated DNA sequence upstream of the coding region that contains the binding site for RNA polymerase II and initiates transcription of the DNA. The promoter region may also include other elements that act as regulators of gene expression.

A “protoplast” is an isolated plant cell without a cell wall or with only part of the cell wall.

“Regulatory elements” refer to sequences involved in controlling the expression of a nucleotide sequence. Regulatory elements comprise a promoter operably linked to the nucleotide sequence of interest and termination signals. They also typically encompass sequences required for proper translation of the nucleotide sequence.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g. a bacterium or plant, which does not contain the heterologous nucleic acid molecule.

A fused peptide of the invention may be provided in an expression cassette for expression of a nucleotide encoding the fused peptide in a host cell of interest, e.g. a plant cell or a microbe. By “plant expression cassette” is intended a DNA construct that is capable of resulting in the expression of a fused peptide from an open reading frame in a plant cell. Typically, these contain a promoter and a coding sequence. Often, such constructs will also contain a 3′ untranslated region. Such constructs may contain a “signal sequence” or “leader sequence” to facilitate co-translational or post-translational transport of the peptide to certain intracellular structures such as the chloroplast (or other plastid), endoplasmic reticulum, or Golgi apparatus. In another embodiment fused or operably fused also relates to an indirect fusion, meaning that all the listed insect viral coat proteins (see SEQ ID Nos 1-4) can also be fused with a binding protein, which then binds the insect trait non-covalently, for example through dsRNA-binding domain (dsRBD) or through other protein domains specifically binding the insect trait dsRNA or chemical, like dsRNA-binding domain (dsRBD) from the human protein kinase R (PKR) as described in “Accelerated delivery of dsRNA in lepidopteran midgut cells by a Galanthus nivalis lectin (GNA)-dsRNA-binding domain fusion protein, Pesticide Biochemistry and Physiology Open Access Volume 175 June 2021” or antibodies or aptamers for binding chemicals with high specificity.

By “signal sequence” is intended a sequence that is known or suspected to result in co-translational or post-translational peptide transport across the cell membrane. In eukaryotes, this typically involves secretion into the Golgi apparatus, with some resulting glycosylation. Insecticidal toxins of bacteria are often synthesized as protoxins, which are proteolytically activated in the gut of the target pest (Chang (1987) Methods Enzymol. 153:507-516). In some embodiments of the present invention, the signal sequence is located in the native sequence, or may be derived from a sequence of the invention. By “leader sequence” is intended any sequence that when translated, results in an amino acid sequence sufficient to trigger co-translational transport of the peptide chain to a subcellular organelle. Thus, this includes leader sequences targeting transport and/or glycosylation by passage into the endoplasmic reticulum, passage to vacuoles, plastids including chloroplasts, mitochondria, and the like. Thus, further provided herein is a polypeptide comprising an amino acid sequence of the present invention that is operably linked to a heterologous leader or signal sequence.

By “plant transformation vector” is intended a DNA molecule that is necessary for efficient transformation of a plant cell. Such a molecule may consist of one or more plant expression cassettes comprising a fused peptide, and may be organized into more than one “vector” DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a nucleic acid construct designed for transfer between different host cells. “Expression vector” refers to a vector that has the ability to incorporate, integrate and express heterologous DNA sequences or fragments in a foreign cell. The cassette will include 5′ and/or 3′ regulatory sequences operably linked to a sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. In some embodiments, the nucleotide sequence is operably linked to a heterologous promoter capable of directing expression of said nucleotide sequence in a host cell, such as a microbial host cell or a plant host cell. The cassette may additionally contain at least one additional gene to be co-transformed into the organism. Alternatively, the additional gene(s) can be provided on multiple expression cassettes.

In various embodiments, the nucleotide sequence encoding a fused peptide of the invention is operably linked to a heterologous promoter capable of directing expression of the nucleotide sequence in a cell, e.g., in a plant cell or a microbe.

The expression cassette will include in the 5′-3′ direction of transcription, a transcriptional and translational initiation region (i.e., a promoter), a DNA sequence encoding a fused peptide of the invention, and a translational and transcriptional termination region (i.e., termination region) functional in plants. The promoter may be native or analogous, or foreign or heterologous, to the plant host and/or to the DNA sequence of the invention. Additionally, the promoter may be the natural sequence or alternatively a synthetic sequence. Where the promoter is “native” or “homologous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds (1987) Nucleic Acids Res. 15:2343-2361. Also, the location of the promoter relative to the transcription start may be optimized. See, e.g., Roberts et al. (1979) Proc. Natl. Acad. Sci. USA, 76:760-764. Many suitable promoters for use in plants are well known in the art.

For instance, suitable constitutive promoters for use in plants include: the promoters from plant viruses, such as the peanut chlorotic streak caulimovirus (PC1SV) promoter (U.S. Pat. No. 5,850,019); the 35S promoter from cauliflower mosaic virus (CaMV) (Odell et al. (1985) Nature 313:810-812); the 35S promoter described in Kay et al. (1987) Science 236: 1299-1302; promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171 and U.S. Pat. No. 5,641,876); ubiquitin (Christensen et al. (1989) Plant Mol. Biol. 12:619-632 and Christensen et al. (1992) Plant Mol. Biol. 18:675-689) and Grefen et al. (2010) Plant J, 64:355-365; pEMU (Last et al. (1991) Theor. Appl. Genet. 81:581-588); MAS (Velten et al. (1984) EMBOJ. 3:2723-2730 and U.S. Pat. No. 5,510,474); maize H3 histone (Lepetit et al. (1992)Mol. Gen. Genet. 231:276-285 and Atanassova et al. (1992) Plant J. 2(3):291-300); Brassica napus ALS3 (PCT application WO97/41228); a plant ribulose-biscarboxylase/oxygenase (RuBisCO) small subunit gene; the circovirus (AU 689 311) or the Cassava vein mosaic virus (CsVMV, U.S. Pat. No. 7,053,205); promoters from soybean (Pbdc6 or Pbdc7, described in WO/2014/150449 or ubiquitin 3 promoter described in U.S. Pat. Nos. 7,393,948 and 8,395,021); and promoters of various Agrobacterium genes (see U.S. Pat. Nos. 4,771,002; 5,102,796; 5,182,200; and 5,428,147).

Suitable inducible promoters for use in plants include: the promoter from the ACE1 system which responds to copper (Mett et al. (1993) PNAS 90:4567-4571); the promoter of the maize In2 gene which responds to benzenesulfonamide herbicide safeners (Hershey et al. (1991) Mol. Gen. Genetics 227:229-237 and Gatz et al. (1994)Mol. Gen. Genetics 243:32-38); and the promoter of the Tet repressor from Tn10 (Gatz et al. (1991)Mol. Gen. Genet. 227:229-237). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al. (1991) Proc. Natl. Acad. Sci. USA 88:10421) or the recent application of a chimeric transcription activator, XVE, for use in an estrogen receptor-based inducible plant expression system activated by estradiol (Zuo et al. (2000) Plant J., 24:265-273). Other inducible promoters for use in plants are described in EP 332104, PCT WO 93/21334 and PCT WO 97/06269 which are herein incorporated by reference in their entirety. Promoters composed of portions of other promoters and partially or totally synthetic promoters can also be used. See, e.g., Ni et al. (1995) Plant J. 7:661-676 and PCT WO 95/14098 describing such promoters for use in plants.

In one embodiment of this invention, a promoter sequence specific for particular regions or tissues of plants can be used to express the pesticidal proteins of the invention, such as promoters specific for seeds (Datla, R. et al., 1997, Biotechnology Ann. Rev. 3, 269-296), especially the napin promoter (EP 255 378 A1), the phaseolin promoter, the glutenin promoter, the helianthinin promoter (WO92/17580), the albumin promoter (WO98/45460), the oleosin promoter (WO98/45461), the SATi promoter or the SAT3 promoter (PCT/US98/06978).

Use may also be made of an inducible promoter advantageously chosen from the phenylalanine ammonia lyase (PAL), HMG-CoA reductase (HMG), chitinase, glucanase, proteinase inhibitor (PI), PR1 family gene, nopaline synthase (nos) and vspB promoters (U.S. Pat. No. 5,670,349, Table 3), the HMG2 promoter (U.S. Pat. No. 5,670,349), the apple beta-galactosidase (ABG1) promoter and the apple aminocyclopropane carboxylate synthase (ACC synthase) promoter (WO98/45445). Multiple promoters can be used in the constructs of the invention, including in succession.

The promoter may include, or be modified to include, one or more enhancer elements. In some embodiments, the promoter may include a plurality of enhancer elements. Promoters containing enhancer elements provide for higher levels of transcription as compared to promoters that do not include them. Suitable enhancer elements for use in plants include the PC1SV enhancer element (U.S. Pat. No. 5,850,019), the CaMV 35S enhancer element (U.S. Pat. Nos. 5,106,739 and 5,164,316) and the FMV enhancer element (Maiti et al. (1997) Transgenic Res. 6:143-156); the translation activator of the tobacco mosaic virus (TMV) described in Application WO87/07644, or of the tobacco etch virus (TEV) described by Carrington & Freed 1990, J. Virol. 64: 1590-1597, for example, or introns such as the adhI intron of maize or intron 1 of rice actin. See also PCT WO96/23898, WO2012/021794, WO2012/021797, WO2011/084370, and WO2011/028914.

Often, such constructs can contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. By “signal sequence” is intended a sequence that is known or suspected to result in cotranslational or post-translational peptide transport across the cell membrane. In eukaryotes, this typically involves secretion into the Golgi apparatus, with some resulting glycosylation. By “leader sequence” is intended any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. It may also be preferable to engineer the plant expression cassette to contain an intron, such that mRNA processing of the intron is required for expression.

By “3′ untranslated region” is intended a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. By “5′ untranslated region” is intended a polynucleotide located upstream of a coding sequence.

Other upstream or downstream untranslated elements include enhancers. Enhancers are polynucleotides that act to increase the expression of a promoter region. Enhancers are well known in the art and include, but are not limited to, the SV40 enhancer region and the 35S enhancer element.

The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Convenient termination regions are available from the Ti-plasmid of A. tumefaciens, such as the octopine synthase and nopaline synthase termination regions. See also Guerineau et al. (1991)Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed host cell (synthetic DNA sequence). That is, the genes can be synthesized using host cell-preferred codons for improved expression, or may be synthesized using codons at a host-preferred codon usage frequency. Expression of the open reading frame of the synthetic DNA sequence in a cell results in production of the polypeptide of the invention. Synthetic DNA sequences can be useful to simply remove unwanted restriction endonuclease sites, to facilitate DNA cloning strategies, to alter or remove any potential codon bias, to alter or improve GC content, to remove or alter alternate reading frames, and/or to alter or remove intron/exon splice recognition sites, polyadenylation sites, Shine-Delgarno sequences, unwanted promoter elements and the like that may be present in a native DNA sequence. Generally, the GC content of the gene will be increased. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, U.S. Patent Publication No. 20090137409, and Murray et al. (1989) Nucleic Acids Res. 17:477-498, herein incorporated by reference.

It is also possible that synthetic DNA sequences may be utilized to introduce other improvements to a DNA sequence, such as introduction of an intron sequence, creation of a DNA sequence that in expressed as a protein fusion to organelle targeting sequences, such as chloroplast transit peptides, apoplast/vacuolar targeting peptides, or peptide sequences that result in retention of the resulting peptide in the endoplasmic reticulum. Thus, in one embodiment, the pesticidal protein is targeted to the chloroplast for expression. In this manner, where the pesticidal protein is not directly inserted into the chloroplast, the expression cassette will additionally contain a nucleic acid encoding a transit peptide to direct the pesticidal protein to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The pesticidal gene to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

### Plant Transformation

Methods of the invention involve introducing a nucleotide construct into a plant. By “introducing” is intended to present to the plant the nucleotide construct in such a manner that the construct gains access to the interior of a cell of the plant. The methods of the invention do not require that a particular method for introducing a nucleotide construct to a plant is used, only that the nucleotide construct gains access to the interior of at least one cell of the plant. Methods for introducing nucleotide constructs into plants are known in the art including, but not limited to, stable transformation methods, transient transformation methods, and virus-mediated methods.

By “plant” is intended whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

The transgenic plants of the invention express one or more of the novel fusion peptides disclosed herein. In some embodiments, the protein or nucleotide sequence of the invention is advantageously combined in plants with other genes which encode proteins or RNAs that confer useful agronomic properties to such plants. Among the genes which encode proteins or RNAs that confer useful agronomic properties on the transformed plants, mention can be made of the DNA sequences encoding proteins which confer tolerance to one or more herbicides, and others which confer tolerance to certain insects, those which confer tolerance to certain diseases, DNAs that encodes RNAs that provide nematode or insect control, and the like. Such genes are in particular described in published PCT Patent Applications WO91/02071 and WO95/06128 and in U.S. Pat. No. 7,923,602 and US Patent Application Publication No. 20100166723, each of which is herein incorporated by reference in its entirety.

Among the DNA sequences encoding proteins which confer tolerance to certain herbicides on the transformed plant cells and plants, mention can be made of a bar or PAT gene or the Streptomyces coelicolor gene described in WO2009/152359 which confers tolerance to glufosinate herbicides, a gene encoding a suitable EPSPS which confers tolerance to herbicides having EPSPS as a target, such as glyphosate and its salts (U.S. Pat. Nos. 4,535,060, 4,769,061, 5,094,945, 4,940,835, 5,188,642, 4,971,908, 5,145,783, 5,310,667, 5,312,910, 5,627,061, 5,633,435), a gene encoding glyphosate-n-acetyltransferase (for example, U.S. Pat. Nos. 8,222,489, 8,088,972, 8,044,261, 8,021,857, 8,008,547, 7,999,152, 7,998,703, 7,863,503, 7,714,188, 7,709,702, 7,666,644, 7,666,643, 7,531,339, 7,527,955, and 7,405,074), a gene encoding glyphosate oxydoreductase (for example, U.S. Pat. No. 5,463,175), or a gene encoding an HPPD inhibitor-tolerant protein (for example, the HPPD inhibitor tolerance genes described in WO 2004/055191, WO 199638567, U.S. Pat. No. 6,791,014, WO2011/068567, WO2011/076345, WO2011/085221, WO2011/094205, WO2011/068567, WO2011/094199, WO2011/094205, WO2011/145015, WO2012/056401, and WO2014/043435).

Among the DNA sequences encoding a suitable EPSPS which confer tolerance to the herbicides which have EPSPS as a target, mention will more particularly be made of the gene which encodes a plant EPSPS, in particular maize EPSPS, particularly a maize EPSPS which comprises two mutations, particularly a mutation at amino acid position 102 and a mutation at amino acid position 106 (WO2004/074443), and which is described in U.S. Pat. No. 6,566,587, hereinafter named double mutant maize EPSPS or 2mEPSPS, or the gene which encodes an EPSPS isolated from Agrobacterium and which is described by sequence ID No. 2 and sequence ID No. 3 of U.S. Pat. No. 5,633,435, also named CP4.

Among the DNA sequences encoding a suitable EPSPS which confer tolerance to the herbicides which have EPSPS as a target, mention will more particularly be made of the gene which encodes an EPSPS GRG23 from Arthrobacter globiformis, but also the mutants GRG23 ACE1, GRG23 ACE2, or GRG23 ACE3, particularly the mutants or variants of GRG23 as described in WO2008/100353, such as GRG23(ace3)R173K of SEQ ID No. 29 in WO2008/100353.

In the case of the DNA sequences encoding EPSPS, and more particularly encoding the above genes, the sequence encoding these enzymes is advantageously preceded by a sequence encoding a transit peptide, in particular the “optimized transit peptide” described in U.S. Pat. No. 5,510,471 or 5,633,448.

Exemplary herbicide tolerance traits that can be combined with the nucleic acid sequence of the invention further include at least one ALS (acetolactate synthase) inhibitor (WO2007/024782); a mutated Arabidopsis ALS/AHAS gene (U.S. Pat. No. 6,855,533); genes encoding 2,4-D-monooxygenases conferring tolerance to 2,4-D (2,4-dichlorophenoxyacetic acid) by metabolization (U.S. Pat. No. 6,153,401); and, genes encoding Dicamba monooxygenases conferring tolerance to dicamba (3,6-dichloro-2-methoxybenzoic acid) by metabolization (US 2008/0119361 and US 2008/0120739).

In various embodiments, the nucleic acid of the invention is stacked with one or more herbicide tolerant genes, including one or more HPPD inhibitor herbicide tolerant genes, and/or one or more genes tolerant to glyphosate and/or glufosinate.

Among the DNA sequences encoding pesticidal proteins concerning properties of tolerance to insects, mention will more particularly be made of the Bt proteins widely described in the literature and well known to those skilled in the art. Mention will also be made of proteins extracted from bacteria such as Photorhabdus (WO97/17432 & WO98/08932).

Among such DNA sequences encoding pesticidal proteins of interest that may be utilized in fusion VLPs or encapsulated VLPs of the invention, which confer novel properties of tolerance to insects comprise, Bt Cry or VIP proteins widely described in the literature and well known to those skilled in the art. These include the Cry1F protein or hybrids derived from a Cry1F protein (e.g., the hybrid Cry1A-Cry1F proteins described in U.S. Pat. Nos. 6,326,169; 6,281,016; 6,218,188, or toxic fragments thereof), the Cry1A-type proteins or toxic fragments thereof, preferably the Cry1Ac protein or hybrids derived from the Cry1Ac protein (e.g., the hybrid Cry1Ab-Cry1Ac protein described in U.S. Pat. No. 5,880,275) or the Cry1Ab or Bt2 protein or insecticidal fragments thereof as described in EP451878, the Cry2Ae, Cry2Af or Cry2Ag proteins as described in WO2002/057664 or toxic fragments thereof, the Cry1A.105 protein described in WO 2007/140256 (SEQ ID No. 7) or a toxic fragment thereof, the VIP3Aa19 protein of NCBI accession ABG20428, the VIP3Aa20 protein of NCBI accession ABG20429 (SEQ ID No. 2 in WO 2007/142840), the VIP3A proteins produced in the COT202 or COT203 cotton events (WO2005/054479 and WO2005/054480, respectively), the Cry proteins as described in WO2001/47952, the VIP3Aa protein or a toxic fragment thereof as described in Estruch et al. (1996), Proc Natl Acad Sci USA. 28; 93(11):5389-94 and U.S. Pat. No. 6,291,156, the insecticidal proteins from Xenorhabdus (as described in WO98/50427), Serratia (particularly from S. entomophila) or Photorhabdus species strains, such as Tc-proteins from Photorhabdus as described in WO98/08932 (e.g., Waterfield et al., 2001, Appl Environ Microbiol. 67(11):5017-24; Ffrench-Constant and Bowen, 2000, Cell Mol Life Sci.; 57(5):828-33). Also any variants or mutants of any one of these proteins differing in some (1-10, preferably 1-5) amino acids from any of the above sequences, particularly the sequence of their toxic fragment, or which are fused to a transit peptide, such as a plastid transit peptide, or another protein or peptide, is included herein.

In yet another embodiment, the pesticidal proteins that may be used in aspects of the invention encompassed herein are MTX-like sequences. The term “MTX” is used in the art to delineate a set of pesticidal proteins that are produced by Bacillus sphaericus. The first of these, often referred to in the art as MTX1, is synthesized as a parasporal crystal which is toxic to mosquitoes. The major components of the crystal are two proteins of 51 and 42 kDa, Since the presence of both proteins are required for toxicity, MTX1 is considered a “binary” toxin (Baumann et al. (1991) Microbiol. Rev. 55:425-436).

By analysis of different Bacillus sphaericus strains with differing toxicities, two new classes of MTX toxins have been identified. MTX2 and MTX3 represent separate, related classes of pesticidal toxins that exhibit pesticidal activity. See, for example, Baumann et al. (1991) Microbiol. Rev. 55:425-436, herein incorporated by reference in its entirety. MTX2 is a 100-kDa toxin. More recently MTX3 has been identified as a separate toxin, though the amino acid sequence of MTX3 from B. sphaericus is 38% identical to the MTX2 toxin of B. sphaericus SSII-1 (Liu, et al. (1996) Appl. Environ. Microbiol. 62: 2174-2176). MTX toxins may be useful for both increasing the insecticidal activity of B. sphaericus strains and managing the evolution of resistance to the Bin toxins in mosquito populations (Wirth et al. (2007) Appl Environ Microbiol 73(19):6066-6071).

In various embodiments, the nucleic acid of the invention can be combined in plants with one or more genes conferring a desirable trait, such as herbicide tolerance, insect tolerance, drought tolerance, nematode control, water use efficiency, nitrogen use efficiency, improved nutritional value, disease resistance, improved photosynthesis, improved fiber quality, stress tolerance, improved reproduction, and the like.

Particularly useful transgenic events which may be combined with the genes of the current invention in plants of the same species (e.g., by crossing or by re-transforming a plant containing another transgenic event with a chimeric gene of the invention), include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946 Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control—herbicide tolerance, deposited as PTA-6233, described in WO2005/103266 or US-A 2005-216969); Event 3006-210-23 (cotton, insect control—herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control—herbicide tolerance, deposited as ATCC PTA-11508, described in WO 11/075593); Event 43A47 (corn, insect control—herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMWB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480);); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.1 (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control—herbicide tolerance, deposited as ATCC PTA 11384, described in US-A 2006-070139); Event DAS-59132 (corn, insect control—herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-1 (brinjal, insect control, not deposited, described in WO 07/091277); Event FI117 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO 98/044140); Event GHB119 (cotton, insect control—herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMWB 41158 or NCIMB 41159, described in US-A 2004-172669 or WO 2004/074492); Event JOPLINI (wheat, disease tolerance, not deposited, described in US-A 2008-064032); Event LL27 (soybean, herbicide tolerance, deposited as NCIMWB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/026345); Event LLRice62 (rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control—herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MS11 (oilseed rape, pollination control—herbicide tolerance, deposited as ATCC PTA-850 or PTA-2485, described in WO2001/031042); Event MS8 (oilseed rape, pollination control—herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control—herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control—herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control—herbicide tolerance, not deposited, described in US-A 2005-039226 or WO2004/099447); Event VIP1034 (corn, insect control—herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession N^{o} PTA-11041) optionally stacked with event EE-GM1/LL27 or event EE-GM2/LL55 (WO2011/063413A2), event DAS-68416-4 (soybean, herbicide tolerance, ATCC Accession N^{o} PTA-10442, WO2011/066360A1), event DAS-68416-4 (soybean, herbicide tolerance, ATCC Accession N^{o} PTA-10442, WO2011/066384A1), event DP-040416-8 (corn, insect control, ATCC Accession N^{o} PTA-11508, WO2011/075593A1), event DP-043A47-3 (corn, insect control, ATCC Accession N^{o} PTA-11509, WO2011/075595A1), event DP-004114-3 (corn, insect control, ATCC Accession N^{o} PTA-11506, WO2011/084621A1), event DP-032316-8 (corn, insect control, ATCC Accession N^{o} PTA-11507, WO2011/084632A1), event MON-88302-9 (oilseed rape, herbicide tolerance, ATCC Accession N^{o} PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession N^{o}. PTA-10296, WO2012/051199A2), event DAS-44406-6 (soybean, stacked herbicide tolerance, ATCC Accession N^{o}. PTA-11336, WO2012/075426A1), event DAS-14536-7 (soybean, stacked herbicide tolerance, ATCC Accession N^{o}. PTA-11335, WO2012/075429A1), event SYN-000H2-5 (soybean, herbicide tolerance, ATCC Accession N^{o}. PTA-11226, WO2012/082548A2), event DP-061061-7 (oilseed rape, herbicide tolerance, no deposit N^{o} available, WO2012071039A1), event DP-073496-4 (oilseed rape, herbicide tolerance, no deposit N^{o} available, US2012131692), event 8264.44.06.1 (soybean, stacked herbicide tolerance, Accession N^{o} PTA-11336, WO2012075426A2), event 8291.45.36.2 (soybean, stacked herbicide tolerance, Accession N^{o}. PTA-11335, WO2012075429A2), event SYHT0H2 (soybean, ATCC Accession N^{o}. PTA-11226, WO2012/082548A2), event MON88701 (cotton, ATCC Accession N^{o} PTA-11754, WO2012/134808A1), event KK179-2 (alfalfa, ATCC Accession N^{o} PTA-11833, WO2013/003558A1), event pDAB8264.42.32.1 (soybean, stacked herbicide tolerance, ATCC Accession N^{o} PTA-11993, WO2013/010094A1), event MZDTO9Y (corn, ATCC Accession N^{o} PTA-13025, WO2013/012775A1).

Transformation of plant cells can be accomplished by one of several techniques known in the art. The pesticidal gene of the invention may be modified to obtain or enhance expression in plant cells. Typically, a construct that expresses such a protein would contain a promoter to drive transcription of the gene, as well as a 3′ untranslated region to allow transcription termination and polyadenylation. The organization of such constructs is well known in the art. In some instances, it may be useful to engineer the gene such that the resulting peptide is secreted, or otherwise targeted within the plant cell. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression.

Typically, this “plant expression cassette” will be inserted into a “plant transformation vector”. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the pesticidal gene are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Transformation protocols as well as protocols for introducing nucleotide sequences into plants may vary depending on the type of plant or plant cell, i.e., monocot or dicot, targeted for transformation. Generation of transgenic plants may be performed by one of several methods, including, but not limited to, microinjection, electroporation, direct gene transfer, introduction of heterologous DNA by Agrobacterium into plant cells (Agrobacterium-mediated transformation), bombardment of plant cells with heterologous foreign DNA adhered to particles, ballistic particle acceleration, aerosol beam transformation (U.S. Published Application No. 20010026941; U.S. Pat. No. 4,945,050; International Publication No. WO 91/00915; U.S. Published Application No. 2002015066), LecI transformation, and various other non-particle direct-mediated methods to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Molecular and biochemical methods can then be used to confirm the presence of the integrated heterologous gene of interest into the genome of the transgenic plant.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

### Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning. A Laboratory Manual. Cold Spring Harbor Laboratory Press, Cold Spring Harbor, NY). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformant, fractionated in a formaldehyde agarose gel, and blotted onto a nylon filter according to standard procedures that are routinely used in the art (Sambrook and Russell, 2001, supra). Expression of RNA encoded by the pesticidal gene is then tested by hybridizing the filter to a radioactive probe derived from a pesticidal gene, by methods known in the art (Sambrook and Russell, 2001, supra).

Western blot, biochemical assays and the like may be carried out on the transgenic plants to confirm the presence of protein encoded by the pesticidal gene by standard procedures (Sambrook and Russell, 2001, supra) using antibodies that bind to one or more epitopes present on the pesticidal protein.

### Pesticidal Activity in Plants

In another aspect of the invention, one may generate transgenic plants expressing a fusion VLPs or encapsulated VLPs comprising a pesticidal protein. Methods described above by way of example may be utilized to generate transgenic plants, but the manner in which the transgenic plant cells are generated is not critical to this invention. Methods known or described in the art such as Agrobacterium-mediated transformation, biolistic transformation, and non-particle-mediated methods may be used at the discretion of the experimenter. Plants expressing a pesticidal protein may be isolated by common methods described in the art, for example by transformation of callus, selection of transformed callus, and regeneration of fertile plants from such transgenic callus. In such process, one may use any gene as a selectable marker so long as its expression in plant cells confers ability to identify or select for transformed cells.

A number of markers have been developed for use with plant cells, such as resistance to chloramphenicol, the aminoglycoside G418, hygromycin, or the like. Other genes that encode a product involved in chloroplast metabolism may also be used as selectable markers. For example, genes that provide resistance to plant herbicides such as glyphosate, bromoxynil, or imidazolinone may find particular use. Such genes have been reported (Stalker et al. (1985) J Biol. Chem. 263:6310-6314 (bromoxynil resistance nitrilase gene); and Sathasivan et al. (1990) Nucl. Acids Res. 18:2188 (AHAS imidazolinone resistance gene). Additionally, the genes disclosed herein are useful as markers to assess transformation of bacterial or plant cells. Methods for detecting the presence of a transgene in a plant, plant organ (e.g., leaves, stems, roots, etc.), seed, plant cell, propagule, embryo or progeny of the same are well known in the art. In one embodiment, the presence of the transgene is detected by testing for pesticidal activity.

Fertile plants expressing fusion VLPs or encapsulated VLPs comprising a pesticidal protein may be tested for pesticidal activity, and the plants showing optimal activity selected for further breeding. Methods are available in the art to assay for pest activity. Generally, the protein is mixed and used in feeding assays. See, for example Marrone et al. (1985) J. of Economic Entomology 78:290-293.

The present invention may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

### Use in Pesticidal Control

General methods for employing strains comprising a nucleotide sequence encoding a fused peptide of the present invention, or a variant thereof, in pest control or in engineering other organisms as pesticidal agents are known in the art. See, for example U.S. Pat. No. 5,039,523 and EP 0480762A2.

Alternatively, the pesticide is produced by introducing a pesticidal gene encoding a fused peptide into a cellular host. Expression of the pesticidal gene results, directly or indirectly, in the intracellular production and maintenance of the pesticidal protein. In one aspect of this invention, these cells are then treated under conditions that prolong the activity of the toxin produced in the cell when the cell is applied to the environment of the target pest(s). The resulting product retains the toxicity of the toxin. These naturally encapsulated pesticidal protein may then be formulated in accordance with conventional techniques for application to the environment hosting a target pest, e.g., soil, water, and foliage of plants. See, for example EPA 0192319, and the references cited therein. Alternatively, one may formulate the cells expressing a gene of this invention such as to allow application of the resulting material as a pesticide.

The active ingredients of the present invention are normally applied in the form of compositions and can be applied to the crop area or plant to be treated, simultaneously or in succession, with other compounds. These compounds can be fertilizers, weed killers, cryoprotectants, surfactants, detergents, pesticidal soaps, dormant oils, polymers, and/or time-release or biodegradable carrier formulations that permit long-term dosing of a target area following a single application of the formulation. They can also be selective herbicides, chemical insecticides, virucides, microbicides, amoebicides, pesticides, fungicides, bacteriocides, nematocides, molluscicides or mixtures of several of these preparations, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise, the formulations may be prepared into edible “baits” or fashioned into pest “traps” to permit feeding or ingestion by a target pest of the pesticidal formulation.

Methods of applying an active ingredient of the present invention or an agrochemical composition of the present invention that contains at least one of the pesticidal proteins produced by the present invention include leaf application, seed coating and soil application. The number of applications and the rate of application depend on the intensity of infestation by the corresponding pest.

The composition may be formulated as a powder, dust, pellet, granule, spray, emulsion, colloid, solution, or such like, and may be prepared by such conventional means as desiccation, lyophilization, homogenation, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

Lepidopteran, hemipteran, dipteran, or coleopteran pests may be killed or reduced in numbers in a given area by the methods of the invention, or may be prophylactically applied to an environmental area to prevent infestation by a susceptible pest. Preferably the pest ingests, or is contacted with, a pesticidally-effective amount of the polypeptide. By “pesticidally-effective amount” is intended an amount of the pesticide that is able to bring about death to at least one pest, or to noticeably reduce pest growth, feeding, or normal physiological development. For example, the pesticide may result in reduced egg hatching, mortality at any stage of development of the insect, reduced molting, and/or reduced feeding of the pest on a target organisms (e.g., reduced number of feeding sites a plant or plant cell and/or reduced damage to a plant or plant cell). This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. The formulations may also vary with respect to climatic conditions, environmental considerations, and/or frequency of application and/or severity of pest infestation.

The pesticide compositions described may be made by formulating either the bacterial cell, the crystal and/or the spore suspension, or the isolated protein component with the desired agriculturally-acceptable carrier. The compositions may be formulated prior to administration in an appropriate means such as lyophilized, freeze-dried, desiccated, or in an aqueous carrier, medium or suitable diluent, such as saline or other buffer. The formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. The term “agriculturally-acceptable carrier” covers all adjuvants, inert components, dispersants, surfactants, tackifiers, binders, etc. that are ordinarily used in pesticide formulation technology; these are well known to those skilled in pesticide formulation. The formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, herein incorporated by reference.

“Pest” includes but is not limited to, insects, fungi, bacteria, nematodes, mites, ticks, and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Coleoptera, Lepidoptera, and Diptera.

The order Coleoptera includes the suborders Adephaga and Polyphaga. Suborder Adephaga includes the superfamilies Caraboidea and Gyrinoidea, while suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

Hemipteran pests (which include species that are designated as Hemiptera, Homoptera, or Heteroptera) include, but are not limited to, Lygus spp., such as Western tarnished plant bug (Lygus hesperus), the tarnished plant bug (Lygus lineolaris), and green plant bug (Lygus elisus); aphids, such as the green peach aphid (Myzus persicae), cotton aphid (Aphis gossypii), cherry aphid or black cherry aphid (Myzus cerasi), soybean aphid (Aphis glycines Matsumura); brown plant hopper (Nilaparvata lugens), and rice green leafhopper (Nephotettix spp.); and stink bugs, such as green stink bug (Acrosternum hilare), brown marmorated stink bug (Halyomorpha halys), southern green stink bug (Nezara viridula), rice stink bug (Oebalus pugnax), forest bug (Pentatoma rufipes), European stink bug (Rhaphigaster nebulosa), and the shield bug Troilus luridus.

Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Spodoptera cosmioides; Spodoptera eridania; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rige: Diatraea saccharalis, sugarcane borer; Spodoptera frugiperda, fall armyworm; Spodoptera cosmioides; Spodoptera eridania; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Chilu suppressalis, Asiatic rice borer; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Spodoptera cosmioides; Spodoptera eridania; Heliothis virescens, cotton budworm; Helicoverpa zea, cotton bollworm; Epilachna varivestis, Mexican bean beetle; Myruspersicae, green peach aphid; Empoascafabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Bareyv: Ostrinia nubilalis, European corn borer; Agrotis ipsilon, black cutworm; Schizaphis graminum, greenbug; Blissus leucopterus, chinch bug; Acrosternum hilare, green stink bug; Euschistus servus, brown stink bug; Euschistus heros, neotropical brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

### Methods for Increasing Plant Yield

Methods for increasing plant yield are provided. The methods comprise providing a plant or plant cell expressing a polynucleotide encoding fusion VLPs or encapsulated VLPs comprising a pesticidal protein disclosed herein and growing the plant or a seed thereof in a field infested with (or susceptible to infestation by) a pest against which said pesticidal protein as pesticidal activity against. In some embodiments, fusion VLPs or encapsulated VLPs comprising a pesticidal protein has pesticidal activity against a lepidopteran, coleopteran, dipteran, hemipteran, or nematode pest, and said field is infested with a lepidopteran, hemipteran, coleopteran, dipteran, or nematode pest. As defined herein, the “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not expressing the pesticidal sequence. In specific methods, plant yield is increased as a result of improved pest resistance of a plant expressing a pesticidal protein disclosed herein. Expression of the pesticidal protein results in a reduced ability of a pest to infest or feed.

The plants can also be treated with one or more chemical compositions, including one or more herbicide, insecticides, or fungicides. Exemplary chemical compositions include: Fruits/Vegetables Herbicides: Atrazine, Bromacil, Diuron, Glyphosate, Linuron, Metribuzin, Simazine, Trifluralin, Fluazifop, Glufosinate, Halosulfuron Gowan, Paraquat, Propyzamide, Sethoxydim, Butafenacil, Halosulfuron, Indaziflam; Fruits/Vegetables Insecticides:

Aldicarb, Bacillus thuringiensis, Carbaryl, Carbofuran, Chlorpyrifos, Cypermethrin, Deltamethrin, Abamectin, Cyfluthrin/beta-cyfluthrin, Esfenvalerate, Lambda-cyhalothrin, Acequinocyl, Bifenazate, Methoxyfenozide, Novaluron, Chromafenozide, Thiacloprid, Dinotefuran, Fluacrypyrim, Spirodiclofen, Gamma-cyhalothrin, Spiromesifen, Spinosad, Rynaxypyr, Cyazypyr, Triflumuron, Spirotetramat, Imidacloprid, Flubendiamide, Thiodicarb, Metaflumizone, Sulfoxaflor, Cyflumetofen, Cyanopyrafen, Clothianidin, Thiamethoxam, Spinotoram, Thiodicarb, Flonicamid, Methiocarb, Emamectin-benzoate, Indoxacarb, Fenamiphos, Pyriproxifen, Fenbutatin-oxid; Fruits/Vegetables Fungieides: Ametoctradin, Azoxystrobin, Benthiavalicarb, Boscalid, Captan, Carbendazim, Chlorothalonil, Copper, Cyazofamid, Cyflufenamid, Cymoxanil, Cyproconazole, Cyprodinil, Difenoconazole, Dimetomorph, Dithianon, Fenamidone, Fenhexamid, Fluazinam, Fludioxonil, Fluopicolide, Fluopyram, Fluoxastrobin, Fluxapyroxad, Folpet, Fosetyl, Iprodione, Iprovalicarb, Isopyrazam, Kresoxim-methyl, Mancozeb, Mandipropamid, Metalaxyl/mefenoxam, Metiram, Metrafenone, Myclobutanil, Penconazole, Penthiopyrad, Picoxystrobin, Propamocarb, Propiconazole, Propineb, Proquinazid, Prothioconazole, Pyraclostrobin, Pyrimethanil, Quinoxyfen, Spiroxamine, Sulphur, Tebuconazole, Thiophanate-methyl, Trifloxystrobin; Cereals Herbicides:

2.4-D, Amidosulfuron, Bromoxynil, Carfentrazone-E, Chlorotoluron, Chlorsulfuron, Clodinafop-P, Clopyralid, Dicamba, Diclofop-M, Diflufenican, Fenoxaprop, Florasulam, Flucarbazone-NA, Flufenacet, Flupyrosulfuron-M, Fluroxypyr, Flurtamone, Glyphosate, Iodosulfuron, Ioxynil, Isoproturon, MCPA, Mesosulfuron, Metsulfuron, Pendimethalin, Pinoxaden, Propoxycarbazone, Prosulfocarb, Pyroxsulam, Sulfosulfuron, Thifensulfuron, Tralkoxydim, Triasulfuron, Tribenuron, Trifluralin, Tritosulfuron; Cereals Fungicides: Azoxystrobin, Bixafen, Boscalid, Carbendazim, Chlorothalonil, Cyflufenamid, Cyproconazole, Cyprodinil, Dimoxystrobin, Epoxiconazole, Fenpropidin, Fenpropimorph, Fluopyram, Fluoxastrobin, Fluquinconazole, Fluxapyroxad, Isopyrazam, Kresoxim-methyl, Metconazole, Metrafenone, Penthiopyrad, Picoxystrobin, Prochloraz, Propiconazole, Proquinazid, Prothioconazole, Pyraclostrobin, Quinoxyfen, Spiroxamine, Tebuconazole, Thiophanate-methyl, Trifloxystrobin; Cereals Insecticides: Dimethoate, Lambda-cyhalthrin, Deltamethrin, alpha-Cypermethrin, β-cyfluthrin, Bifenthrin, Imidacloprid, Clothianidin, Thiamethoxam, Thiacloprid, Acetamiprid, Dinetofuran, Clorphyriphos, Pirimicarb, Methiocarb, Sulfoxaflor; Maize Herbicides: Atrazine, Alachlor, Bromoxynil, Acetochlor, Dicamba, Clopyralid, (S-)Dimethenamid, Glufosinate, Glyphosate, Isoxaflutole, (S-)Metolachlor, Mesotrione, Nicosulfuron, Primisulfuron, Rimsulfuron, Sulcotrione, Foramsulfuron, Topramezone, Tembotrione, Saflufenacil, Thiencarbazone, Flufenacet, Pyroxasulfon; Maize Insecticides: Carbofuran, Chlorpyrifos, Bifenthrin, Fipronil, Imidacloprid, Lambda-Cyhalothrin, Tefluthrin, Terbufos, Thiamethoxam, Clothianidin, Spiromesifen, Flubendiamide, Triflumuron, Rynaxypyr, Deltamethrin, Thiodicarb, β-Cyfluthrin, Cypermethrin, Bifenthrin, Lufenuron, Tebupirimphos, Ethiprole, Cyazypyr, Thiacloprid, Acetamiprid, Dinetofuran, Avermectin; Maize Fungicides: Azoxystrobin, Bixafen, Boscalid, Cyproconazole, Dimoxystrobin, Epoxiconazole, Fenitropan, Fluopyram, Fluoxastrobin, Fluxapyroxad, Isopyrazam, Metconazole, Penthiopyrad, Picoxystrobin, Propiconazole, Prothioconazole, Pyraclostrobin, Tebuconazole, Trifloxystrobin; Rice Herbicides: Butachlor, Propanil, Azimsulfuron, Bensulfuron, Cyhalofop, Daimuron, Fentrazamide, Imazosulfuron, Mefenacet, Oxaziclomefone, Pyrazosulfuron, Pyributicarb, Quinclorac, Thiobencarb, Indanofan, Flufenacet, Fentrazamide, Halosulfuron, Oxaziclomefone, Benzobicyclon, Pyriftalid, Penoxsulam, Bispyribac, Oxadiargyl, Ethoxysulfuron, Pretilachlor, Mesotrione, Tefuryltrione, Oxadiazone, Fenoxaprop, Pyrimisulfan; Rice Insecticides: Diazinon, Fenobucarb, Benfuracarb, Buprofezin, Dinotefuran, Fipronil, Imidacloprid, Isoprocarb, Thiacloprid, Chromafenozide, Clothianidin, Ethiprole, Flubendiamide, Rynaxypyr, Deltamethrin, Acetamiprid, Thiamethoxam, Cyazypyr, Spinosad, Spinotoram, Emamectin-Benzoate, Cypermethrin, Chlorpyriphos, Etofenprox, Carbofuran, Benfuracarb, Sulfoxaflor; Rice Fungicides: Azoxystrobin, Carbendazim, Carpropamid, Diclocymet, Difenoconazole, Edifenphos, Ferimzone, Gentamycin, Hexaconazole, Hymexazol, Iprobenfos (IBP), Isoprothiolane, Isotianil, Kasugamycin, Mancozeb, Metominostrobin, Orysastrobin, Pencycuron, Probenazole, Propiconazole, Propineb, Pyroquilon, Tebuconazole, Thiophanate-methyl, Tiadinil, Tricyclazole, Trifloxystrobin, Validamycin; Cotton Herbicides: Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron; Cotton Insecticides: Acephate, Aldicarb, Chlorpyrifos, Cypermethrin, Deltamethrin, Abamectin, Acetamiprid, Emamectin Benzoate, Imidacloprid, Indoxacarb, Lambda-Cyhalothrin, Spinosad, Thiodicarb, Gamma-Cyhalothrin, Spiromesifen, Pyridalyl, Flonicamid Flubendiamide, Triflumuron, Rynaxypyr, Beta-Cyfluthrin, Spirotetramat, Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl](2,2-difluorethyl)amino]furan-2(5H)-on, Thiodicarb, Avermectin, Flonicamid, Pyridalyl, Spiromesifen, Sulfoxaflor; Cotton Fungieides: Azoxystrobin, Bixafen, Boscalid, Carbendazim, Chlorothalonil, Copper, Cyproconazole, Difenoconazole, Dimoxystrobin, Epoxiconazole, Fenamidone, Fluazinam, Fluopyram, Fluoxastrobin, Fluxapyroxad, Iprodione, Isopyrazam, Isotianil, Mancozeb, Maneb, Metominostrobin, Penthiopyrad, Picoxystrobin, Propineb, Prothioconazole, Pyraclostrobin, Quintozene, Tebuconazole, Tetraconazole, Thiophanate-methyl, Trifloxystrobin; Soybean Herbicides: Alachlor, Bentazone, Trifluralin, Chlorimuron-Ethyl, Cloransulam-Methyl, Fenoxaprop, Fomesafen, Fluazifop, Glyphosate, Imazamox, Imazaquin, Imazethapyr, (S-)Metolachlor, Metribuzin, Pendimethalin, Tepraloxydim, Glufosinate; Soybean Insecticides: Lambda-cyhalothrin, Methomyl, Imidacloprid, Clothianidin, Thiamethoxam, Thiacloprid, Acetamiprid, Dinetofuran, Flubendiamide, Rynaxypyr, Cyazypyr, Spinosad, Spinotoram, Emamectin-Benzoate, Fipronil, Ethiprole, Deltamethrin, β-Cyfluthrin, gamma and lambda Cyhalothrin, 4-[[(6-Chlorpyridin-3-yl)methyl](2,2-difluorethyl)amino]furan-2(5H)-on, Spirotetramat, Spinodiclofen, Triflumuron, Flonicamid, Thiodicarb, beta-Cyfluthrin; Sovbean Fungicides: Azoxystrobin, Bixafen, Boscalid, Carbendazim, Chlorothalonil, Copper, Cyproconazole, Difenoconazole, Dimoxystrobin, Epoxiconazole, Fluazinam, Fluopyram, Fluoxastrobin, Flutriafol, Fluxapyroxad, Isopyrazam, Iprodione, Isotianil, Mancozeb, Maneb, Metconazole, Metominostrobin, Myclobutanil, Penthiopyrad, Picoxystrobin, Propiconazole, Propineb, Prothioconazole, Pyraclostrobin, Tebuconazole, Tetraconazole, Thiophanate-methyl, Trifloxystrobin; Sugarbeet Herbicides: Chloridazon, Desmedipham, Ethofumesate, Phenmedipham, Triallate, Clopyralid, Fluazifop, Lenacil, Metamitron, Quinmerac, Cycloxydim, Triflusulfuron, Tepraloxydim, Quizalofop; Sugarbeet Insecticides: Imidacloprid, Clothianidin, Thiamethoxam, Thiacloprid, Acetamiprid, Dinetofuran, Deltamethrin, β-Cyfluthrin, gamma/lambda Cyhalothrin, 4-[[(6-Chlorpyridin-3-yl)methyl](2,2-difluorethyl)amino]furan-2(5H)-on, Tefluthrin, Rynaxypyr, Cyaxypyr, Fipronil, Carbofuran; Canola Herbicides: Clopyralid, Diclofop, Fluazifop, Glufosinate, Glyphosate, Metazachlor, Trifluralin Ethametsulfuron, Quinmerac, Quizalofop, Clethodim, Tepraloxydim; Canola Fungieides: Azoxystrobin, Bixafen, Boscalid, Carbendazim, Cyproconazole, Difenoconazole, Dimoxystrobin, Epoxiconazole, Fluazinam, Fluopyram, Fluoxastrobin, Flusilazole, Fluxapyroxad, Iprodione, Isopyrazam, Mepiquat-chloride, Metconazole, Metominostrobin, Paclobutrazole, Penthiopyrad, Picoxystrobin, Prochloraz, Prothioconazole, Pyraclostrobin, Tebuconazole, Thiophanate-methyl, Trifloxystrobin, Vinclozolin; Canola Insecticides: Carbofuran, Thiacloprid, Deltamethrin, Imidacloprid, Clothianidin, Thiamethoxam, Acetamiprid, Dinetofuran, β-Cyfluthrin, gamma and lambda Cyhalothrin, tau-Fluvaleriate, Ethiprole, Spinosad, Spinotoram, Flubendiamide, Rynaxypyr, Cyazypyr, 4-[[(6-Chlorpyridin-3-yl)methyl](2,2-difluorethyl)amino]furan-2(5H)-on.

Methods of Introducing Gene of the Invention into Another Plant

Also provided herein are methods of introducing the nucleic acid of the invention into another plant. The nucleic acid of the invention encoding a fusion VLPs or encapsulated VLPs, or a fragment thereof, can be introduced into second plant by recurrent selection, backcrossing, pedigree breeding, line selection, mass selection, mutation breeding and/or genetic marker enhanced selection.

Thus, in one embodiment, the methods of the invention comprise crossing a first plant comprising a nucleic acid of the invention with a second plant to produce F1 progeny plants and selecting F1 progeny plants that comprise the nucleic acid of the invention. The methods may further comprise crossing the selected progeny plants with the first plant comprising the nucleic acid of the invention to produce backcross progeny plants and selecting backcross progeny plants that comprise the nucleic acid of the invention. Methods for evaluating pesticidal activity are provided elsewhere herein. The methods may further comprise repeating these steps one or more times in succession to produce selected second or higher backcross progeny plants that comprise the nucleic acid of the invention.

Any breeding method involving selection of plants for the desired phenotype can be used in the method of the present invention. In some embodiments, The F1 plants may be self-pollinated to produce a segregating F2 generation. Individual plants may then be selected which represent the desired phenotype (e.g., pesticidal activity) in each generation (F3, F4, F5, etc.) until the traits are homozygous or fixed within a breeding population.

The second plant can be a plant having a desired trait, such as herbicide tolerance, insect tolerance, drought tolerance, nematode control, water use efficiency, nitrogen use efficiency, improved nutritional value, disease resistance, improved photosynthesis, improved fiber quality, stress tolerance, improved reproduction, and the like. The second plant may be an elite event as described elsewhere herein

In various embodiments, plant parts (whole plants, plant organs (e.g., leaves, stems, roots, etc.), seeds, plant cells, propagules, embryos, and the like) can be harvested from the resulting cross and either propagated or collected for downstream use (such as food, feed, biofuel, oil, flour, meal, etc.).

### Methods of Obtaining a Plant Product

The present invention also relates to a process for obtaining a commodity product, comprising harvesting and/or milling the grains from a crop comprising a nucleic acid of the invention to obtain the commodity product. Agronomically and commercially important products and/or compositions of matter including but not limited to animal feed, commodities, and plant products and by-products that are intended for use as food for human consumption or for use in compositions and commodities that are intended for human consumption, particularly devitalized seed/grain products, including a (semi-)processed products produced from such grain/seeds, wherein said product is or comprises whole or processed seeds or grain, animal feed, corn or soy meal, corn or soy flour, corn, corn starch, soybean meal, soy flour, flakes, soy protein concentrate, soy protein isolates, texturized soy protein concentrate, cosmetics, hair care products, soy nut butter, natto, tempeh, hydrolyzed soy protein, whipped topping, shortening, lecithin, edible whole soybeans (raw, roasted, or as edamame), soy yogurt, soy cheese, tofu, yuba, as well as cooked, polished, steamed, baked or parboiled grain, and the like are intended to be within the scope of the present invention if these products and compositions of matter contain detectable amounts of the nucleotide and/or amino acid sequences set forth herein as being diagnostic for any plant containing such nucleotide sequences.

The following examples are offered by way of illustration and not by way of limitation.

### EXPERIMENTAL EXAMPLES

### Example 1. Discovery and/or Identification of Novel Transport Peptides that can be Utilized as a Fusion VLP or Encapsulated VLP

By way of example four transport peptides were discovered and/or identified which can be utilized in either a fusion VLP or an encapsulation VLP system.

First, applicants characterized a virus member of the genus Totivirus, called NvTv from Nezara viridula stinkbug (see details below as to methodology used to characterize and isolate). Applicants sequenced the genome of this virus and mapped their putative genes (ORFs) including those that encode for putative viral coat proteins (CP) One such NvTv CP is shown in SEQ ID NO: 1 (herein, “NVTV-CP”).

Second, Lepidopteran virus, Helicoverpa armigera stunt virus (HaSV) which infects multiple Heliothine Sp. is a well characterized virus in the literature and the genome of HaSV has been previously annotated (Gordon et al., 1995). HaSV-CP is as demonstrated in SEQ ID NO: 2.

Third, a virus infecting Fall army worm (FAW) was discovered. This is a novel RNA virus in the family Tymoviridae, and is identified as FAW macula-like virus (herein, “SfMLV”). Applicants were able to annotate the genome of SfMLV and identify a putative CP as shown in SEQ ID NO: 3 (herein, “SfMLV-CP”).

Fourth, a FAW virus, Spodoptera frugiperda rhabdovirus (herein, “SfRV”) originally published and listed as a putative glycoprotein (Ma, H. et al. J. Virol 88(12) 6576-6585, 2014). SfRV-CP is represented in SEQ ID NO: 4.

Virus Purification from Adult Nezara viridula and Characterization of NvTV Virus

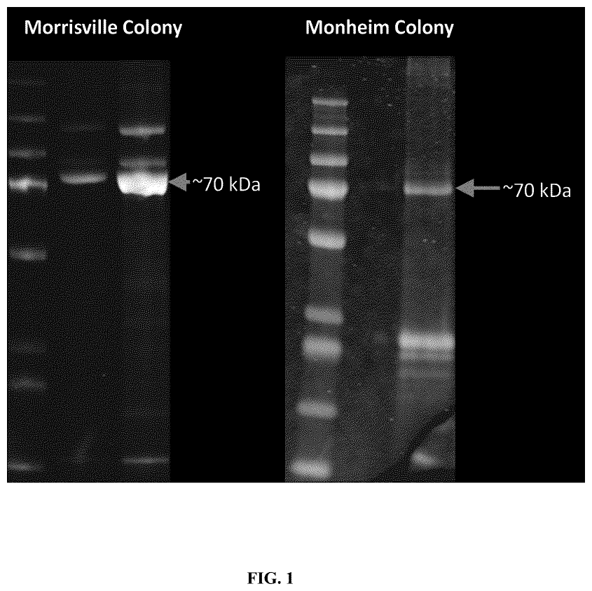

Nezara adults from two colonies (i.e. the Morrisville Colony and the Monheim Colony) were used for virus purification and processed separately. Nezara adults (˜2 g) were ground in liquid nitrogen in a pre-cooled mortar and pestle. The powdered Nezara adults were transferred to a 30 mL centrifuge tube on ice. Sodium phosphate buffer was added to the tube and was briefly vortexed. The tube was shaken vigorously after adding the chloroform and incubated on ice for 5 minutes. The tube was then centrifuged at 8,000 rpm at 4° C. for 25 minutes in a centrifuge. The resulting supernatant was passed through a syringe filter sterilizer (0.45 sM) into a 30 mL centrifuge tube. Sodium phosphate buffer was added to the filtered supernatant. The supernatant was once again centrifuged at 8,000 rpm at 4° C. for 25 minutes in a centrifuge. The resulting supernatant was then transferred to a 30 mL ultracentrifuge tube and centrifuged at 40,000 rpm at 4° C. for 2.5 hours. The supernatant was removed and 5 mL of 0.01M sodium phosphate buffer was added to the pellet. The tube was covered with parafilm and placed on ice at 4° C. overnight with shaking. The re-suspended virus mixture was transferred into 1.5 mL centrifuge tube and centrifuged at 10,000 rpm at 4° C. for 5 minutes. The resulting supernatant was transferred into a new tube. The supernatant was added to the top layer of a 30% sucrose cushion in an ultracentrifuge tube. The tube was centrifuged at 43,000 rpm at 4° C. for 3 hours in an ultracentrifuge. The resulting liquid was removed and the pellet re-suspended overnight at 4° C. as previously described. The stock of purified virus was re-suspended in Sodium phosphate buffer. Both viral preparations were analyzed and confirmed at approximately 70 kDa on SDS-PAGE gels (see FIG. 1).

### Mass Spectrophometry Analysis of Protein Bands for the Identification of Virus

The ˜70 kDa protein bands were cut out form both preparations and sent for Mass spec analysis. Mass sepc data was searched against the denovo assembled Nezara RNA seq data using MASCOT search. The strongest contig hits were used for further analysis to identify the virus using MIDAS blast. It is named as Nezara viridula Totivirus (NvTV) and is a double strand RNA virus in the family Totiviridae. Its genome is 6 kb which encodes a single capsid protein and a RNA dependant RNA poymerase (RdRP). NvTV is present in both the Nezara colonies. It falls into a unclassifed genera with other closely related totiviruses from two ants (Camponotus sp.). The percent homology at the aminoacid level for the RdRP region is 40% and for the Capsid protein is 30%.

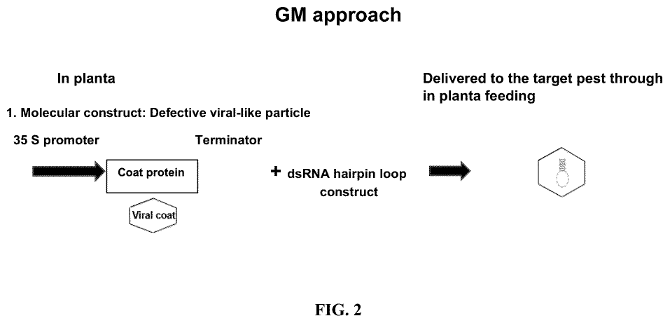

We propose to exploit the viral coat proteins from NvTV, HaSV and SFMLV (named NvTV-CP, HaSV-CP, and SFMLV-CP respectively) and take advantage of their ability to self-assemble into viral-like particles and to transport different pesticidal proteins encapsulated within the CP. This approach can be used as a sprayable application or alternatively through the genetic engineering of plants (see FIG. 2). Note to FIG. 2: To maintain specificity, the CP gene will be selected based on the target species. i.e., if target species is Stink bug sp., a viral coat protein infecting stink bug will be used (e.g. NvTV-CP). If the target species is a lepidopteran, like fall army worm or Heliothine sp., viral CP infecting the specific species will be used (e.g. for FAW, SfMLV). Although this technology could work with viruses that encode multiple coat proteins, we have decided to focus on those viruses that encode for only one CP, like NvTV and HaSV. This approach could be extended to various other viruses that commonly infect insect pests or fungal pathogens. As a sprayable application, NvTV-CP, SfMLV and HaSV-CP can be expressed in vitro and complex them with different cargos like dsRNA, peptides or small molecules.

### Example 2: Cell Expression

NvTV-CP and HaSV-CP proteins were expressed in bacterial, insect cell expression as well as plant expression (Tobacco transient expression) systems. Western blot analysis confirmed expression of NvTV-CP and HaSV-CP in all instances (data not shown).

### Bacterial Expression Construct and Expression of NvTV Coat Protein:

E. coli expression construct (pTR159-NvTV) was used for the expression of full length NvTV coat protein in bacterial expression system. Full length open reading frame (3.2 kb) of NvTV coat protein is fused in frame with maltose binding protein (MBP) under PtacI promoter. Plasmid DNA was transformed into T7 Express E. coli competent cells according to manufacturer's protocol (NEB). 15 ml of overnight grown culture was inoculated into 500 ml of Terrific broth (TB) medium and grown at 37 degree Celsius at 250 rpm until the optical density (OD) reached 0.7-0.8. Cultures were next induced with 1 mM IPTG and grown overnight at 20 degree Celsius at 250 rpm. Following, samples were aliquoted and spun post-induction sample at 13,000 rpm for 5 minutes. Supernatant was removed and freeze thaw pellet and finally run SDS page gel on frozen cell pellets samples.

### Plant Expression Construct and Expression of NvTV Coat Protein in Tobacco Transient System:

Binary plant expression construct (pTR2026) was designed for the expression of full length ORF of NvTV-CP. The NvTV-CP was expressed under the 35S cauliflower mosaic virus promoter. This binary vector was transformed into Agrobacterium competent cells and incubated overnight at 28 degree Celsius. Streak the agrobacterium cells transformed with the binary vector expressing NvTV coat protein from a fresh plate incubated at 28 degree Celsius. Freshly grown agrobacterium cells were resuspended in co-cultivation media with acetosyringone. Agrobacterium cells were resuspended in co-cultivation media to obtain an optical density of 0.5 and incubated at 30 degree Celsius to allow induction. Two fully expanded leaves of a 3-week-old tobacco plant and 2 plants per were infiltrated with the agrobacterium cells. Sample leaf punches within the area of infiltration were collected and the protein was extracted and run on a SDS-PAGE gel and probed with NvTV coat protein antiserum at a dilution of 1:5000. Phenotypic analysis, imaging of the leaves was done on day 2 (before sampling) and on day 5 and 7 for phytotoxicity. Results from SDS-PAGE gel confirmed both expression of the native NvTV-CP (pTR2026) and a soy optimized NvTV-CP (pTR2027). In both instances purified NvTV-CP probed with NvTV antiserum detects 95 and 70 kDa bands.

### Bacterial Expression Construct and Expression of HaSV Coat Protein:

An E. coli expression construct was used for the expression of full length HaSV-CP in bacterial expression system. Full length open reading frame (1.9 kb) of HaSV-CP was fused in frame with maltose binding protein (MBP) under a PtacI promoter. Plasmid DNA was transformed into BL21 Star™ (DE3) cells E. coli competent cells according to manufacturer's protocol (Invitrogen, Massachusetts U.S.). A colony was inoculated in 1 ml of instant Terrific broth (TB) medium and grown at 37 degrees Celsius at 250 rpm until the optical density (OD) reached 0.7-0.8. Cultures were induced with 1 mM IPTG and grown overnight at 20 degrees Celsius at 250 rpm. Next, samples were aliquoted and spun post-induction sample at 13,000 rpm for 5 minutes. Pellets were isolated, samples and ran on a SDS page gel. Cell pellet and supernatant lysates were both analyzed on a SDS page gel and probed with HaSV peptide antibodies at a dilution of 1:15000 and imaged using LICOR image.

### Insect Cell Expression Construct and Expression of HaSV Coat Protein:

Full length ORF of HaSV coat protein was cloned into a commercially available pFastBac-HTA vector under a polyhedrin promoter. Baculovirus expressing HaSV coat protein was generated according to manufacturer's instructions in Bac to Bac expression system (Invitrogen, Massachusetts U.S.). For large scale expression and purification of HaSV Sf9 cells in a 500 ml flask were infected with P2 HaSV virus stock at MOI-3 (Multiplicity of Infection) and incubated for 72 hours. After 72 hours the cells were pelleted and lysed with cell lysis buffer (50 mM Tris; 500 mM NaCl; 5% Glycerol; 1 mM PMSF; 12.5 μg/ml leupeptin; 6.25 μg/ml aprotinin 2.5 μg/ml pepstatin; pH 8.0). Lysate was purified on a His column and washed with buffer (50 mM Tris, pH 8.0 500 mM NaCl; 15% Glycerol; 5 mM Imidazole). Purified HaSV coat protein was eluted in elution buffer (50 mM Tris, pH 8.0; 500 mM NaCl; 15% Glycerol; 500 mM Imidazole. Two micrograms of protein was run on a SDS page gel and probed with a mouse anti-His antibody.

### Example 3 Creation of Encapsulated VLPs**

Virus coat proteins have the ability to encapsulate their own genetic material into their coats due to the presence of sequence specific or cis element like recognizing sequences in their genome.

### Production of Bacmids

Bacmids were produced essentially following the process outlined in the “Bac-to-Bac Baculovirus Expression System” User Guide. Briefly, the SfMLV capsid protein was cloned into either the pFastBac HT A or pFastBac Dual expression vectors following standard techniques. These constructs were then modified as needed to introduce eGFP dsRNA or unagi DNA at the second promoter (pFastBAc Dual) or tags/cargo (pFastBac HT A). DNA was harvested from sequence confirmed clones and transformed into DH10bac E. coli as per manufacturer's instructions. Cells were plated on Bacmid Selection Plates (Molecular Toxicology Catalog #2140S125). White positives were selected and grown overnight in 5 mL of selection media. Bacmids were isolated by EtOH precipitation and evaluated by PCR.

### Virus Production

Cells were transfected with Cellfectin II (ThermoFisher Catalog #10362100) as outlined in the “Bac-to-Bac Baculovirus Expression System” User Guide. Viral production is evaluated by SDS-PAGE/Western Blot of infected cells and supernatant when P1 is harvested. If bands of the correct size are observed 40 uL of the P1 virus is used to infect one well of a 6-Well plate seeded with Sf9 insect cells. The infection is then allowed to continue on until 70-80% of cells are dead which takes 3-5 days. The P2 virus is then collected and used to generate capsids as described.

### Analysis of Capsids

SfMLV-CP was found to package a 950 nucleotide dsRNA hairpin like construct from eGFP RNA efficiently. It was found via qPCR that each capsid from SfMLV-CP contains approximately 1.07 copies of eGFP. A series of enzymatic digests were then undertaken to determine if the observed signal is from packaged RNA, residual RNA, or RNA bound to the outside of the viral particle. The SfMLV RNA loaded capsule was divided into 4 pools and the loaded capsids were subjected to the following treatments prior to mRNA extraction:

### UNT-No Treatment

Proteinase K Treatment: This should digest the capsid and may cause a loss of mRNA, thereby decreasing the observed signal.

DNase Treatment: Will digest any DNA that is attached to the capsid exterior. If exterior DNA is the source of our signal, we will see a decrease or loss of signal following this treatment.

RNase Treatment: This will digest any external or capsid attached mRNA. If the signal source is adherent mRNA this should remove or decrease that signal.

All treatments were conducted as per manufacture's guidelines on pelleted capsids resuspended in the appropriate buffers. Following treatment total RNA was recovered, converted to cDNA and evaluated by qPCR.

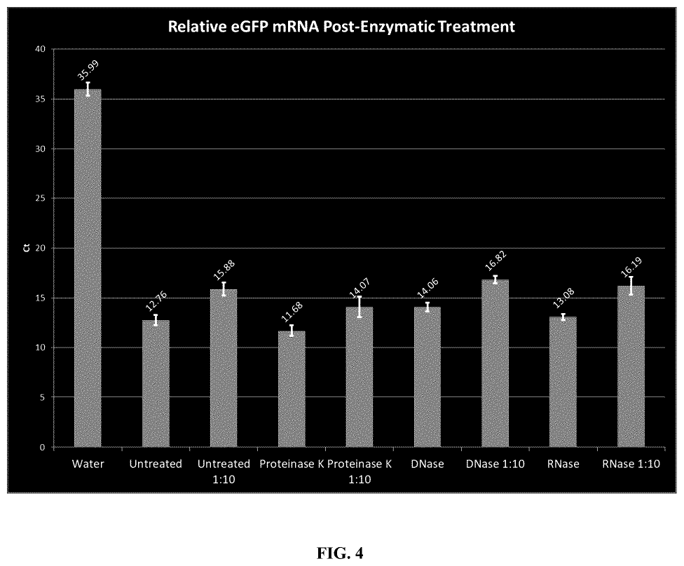

As shown in FIG. 4, all samples gave a strong signal indicative of eGFP mRNA being present inside the capsid.

**Data for Example 3 provided by Dr. Suchetana Mukhopadhyay and David Penn of Indiana University.

### Example 4 Evaluation of Plant Events

Two types of assays can be used for evaluating Soy T0 events. Leaf disc bioassay to test for effectiveness against relative insects and green house trials where fully grown plants expressing the encapsulated VLPs of the invention are subjected to insect feeding and compared to controls.

### Example 5. Soybean Transformation

Soybean transformation is achieved using methods well known in the art, such as the one described using the Agrobacterium tumefaciens mediated transformation soybean half-seed explants using essentially the method described by Paz et al. (2006), Plant cell Rep. 25:206. Transformants are identified using tembotrione as selection marker. The appearance of green shoots was observed, and documented as an indicator of tolerance to the herbicide isoxaflutole or tembotrione. The tolerant transgenic shoots will show normal greening comparable to wild-type soybean shoots not treated with isoxaflutole or tembotrione, whereas wild-type soybean shoots treated with the same amount of isoxaflutole or tembotrione will be entirely bleached. This indicates that the presence of the HPPD protein enables the tolerance to HPPD inhibitor herbicides, like isoxaflutole or tembotrione.

Tolerant green shoots are transferred to rooting media or grafted. Rooted plantlets are transferred to the greenhouse after an acclimation period. Plants containing the transgene are then sprayed with HPPD inhibitor herbicides, as for example with tembotrione at a rate of 100 g AI/ha or with mesotrione at a rate of 300 g AI/ha supplemented with ammonium sulfate methyl ester rapeseed oil. Ten days after the application the symptoms due to the application of the herbicide are evaluated and compared to the symptoms observed on wild type plants under the same conditions.

### Example 6. Transformation of Maize Cells

Maize ears are best collected 8-12 days after pollination. Embryos are isolated from the ears, and those embryos 0.8-1.5 mm in size are preferred for use in transformation. Embryos are plated scutellum side-up on a suitable incubation media, such as DN62A5S media (3.98 g/L N6 Salts; 1 mL/L (of 1000× Stock) N6 Vitamins; 800 mg/L L-Asparagine; 100 mg/L Myo-inositol; 1.4 g/L L-Proline; 100 mg/L Casamino acids; 50 g/L sucrose; 1 mL/L (of 1 mg/mL Stock) 2,4-D). However, media and salts other than DN62A5S are suitable and are known in the art. Embryos are incubated overnight at 25° C. in the dark. However, it is not necessary per se to incubate the embryos overnight.

The resulting explants are transferred to mesh squares (30-40 per plate), transferred onto osmotic media for about 30-45 minutes, then transferred to a beaming plate (see, for example, PCT Publication No. WO/0138514 and U.S. Pat. No. 5,240,842).

DNA constructs designed to the genes of the invention in plant cells are accelerated into plant tissue using an aerosol beam accelerator, using conditions essentially as described in PCT Publication No. WO/0138514. After beaming, embryos are incubated for about 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for about 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

Materials
DN62A5S Media

| Components | Per Liter | | Source |
|---|---|---|---|
| Chu's N6 Basal Salt | 3.98 | g/L | Phytotechnology Labs |
| Mixture (Prod. No. C 416) | | | |
| Chu's N6 Vitamin Solution | 1 mL/L | | Phytotechnology Labs |
| (Prod. No. C 149) | (of 1000× Stock) | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casamino acids | 100 | mg/L | Fisher Scientific |
| Sucrose | 50 | g/L | Phytotechnology Labs |

| | | |
|---|---|---|
| 2,4-D (Prod. No. D-7299) | 1 mL/L | Sigma |
| | (of 1 mg/mL Stock) | |

The pH of the solution is adjusted to pH 5.8 with 1N KOH/1N KCl, Gelrite (Sigma) is added at a concentration up to 3 g/L, and the media is autoclaved. After cooling to 50° C., 2 ml/L of a 5 mg/ml stock solution of silver nitrate (Phytotechnology Labs) is added.

### Example 7. Transformation of Genes or Constructs of the Invention in Plant Cells by Agrobacterium-Mediated Transformation

Ears are best collected 8-12 days after pollination. Embryos are isolated from the ears, and those embryos 0.8-1.5 mm in size are preferred for use in transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors for Ti plasmid mediated transfer for about 5-10 min, and then plated onto co-cultivation media for about 3 days (22° C. in the dark). After co-cultivation, explants are transferred to recovery period media for 5-10 days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art.

What is claimed is:

1. A plant comprising an expression construct wherein said expression construct contains a promoter operably linked to a nucleotide sequence that comprises a nucleotide sequence that encodes a transport peptide operably fused to a cargo molecule.

2. The plant of claim 1, wherein the cargo molecule is selected from the group consisting of a nucleotide sequence encoding a protein, a dsRNA or a nucleotide sequence that confers gene silencing in a cell.

3. The plant of claim 1, wherein the cargo molecule is active against a plant pathogen.

4. The plant of claim 1, wherein the cargo molecule is insecticidal or fungicidal.

5. The plant of claim 4, wherein the cargo molecule is a nucleotide sequence that encodes a binding protein for RNA, dsRNA or for other small molecules with pesticidal activity and wherein the transport protein and the binding protein for RNA, dsRNA or for other small molecules with pesticidal activity form a fusion peptide upon expression in a plant.

6. The plant of claim 4, wherein the cargo molecule is a nucleotide sequence that encodes a pesticidal protein and wherein the transport protein and the cargo molecule form a fusion peptide upon expression in a plant.

7. The plant of claim 6, wherein the pesticidal protein is a Cry, MTX or VIP protein.

8. The plant of claim 1, wherein the transport peptide is derived from a plant pathogen virus.

9. The plant of claim 8, wherein the plant pathogen virus is any virus selected from the group consisting of Totivirus, Omegatetravirus, Tymoviridae, and rhabdovirus.

10. The plant of claim 9, wherein the plant pathogen virus is selected from the any one of the following: Nezara Totivirus, Helicoverpa armigera stunt virus, FAW-macule-like virus or Spodoptera frugiperda rhabdovirus.

11. The plant of claim 1, wherein the nucleotide sequence that encodes a transport peptide encodes a protein having between 70% and 100% sequence identity to any one of SEQ ID Nos: 1-4.

12. The plant of claim 3, wherein the plant pathogen is selected from the group consisting of lepidopteran, coleopteran, dipteran, hemipteran, and nematode pest.

13. The plant of claim 12, wherein the plant pathogen is CEW, SAW, FAW, Helicoverpa armigera or Stink bug.

14. The plant of claim 1, wherein the plant is either a monocot or dicot.

15. The plant of claim 5, wherein the expressed fusion peptide enters the hemocoel of an insect.

16. The plant of claim 15, wherein the expressed fusion peptide kills the insect or decreases the insect's growth.

17. An expression construct wherein said expression construct contains a promoter operably linked to a nucleotide sequence that encodes a transport peptide operably fused to a cargo molecule.

18. The expression construct of claim 17, wherein the cargo molecule is selected from the group consisting of a nucleotide encoding a protein, a dsRNA or a nucleotide that confers gene silencing in a cell.

19. The expression construct of claim 17, wherein the cargo molecule is active against a plant pathogen.

20. The expression construct of claim 17, wherein the cargo molecule is insecticidal or fungicidal.

21. The expression construct of claim 20, wherein the cargo molecule is a nucleotide sequence that encodes a pesticidal protein.

22. The expression construct of claim 21, wherein the pesticidal protein is a Cry, MTX or VIP protein.

23. The expression construct of claim 17, wherein the transport peptide is derived from a plant pathogen virus.

24. The expression construct of claim 23, wherein the plant pathogen virus is any virus selected from the group consisting of Totivirus, Omegatetravirus, Tymoviridae, and rhabdovirus.

25. The expression construct of claim 24, wherein the plant pathogen virus is selected from the any one of the following: Nezara Totivirus, Helicoverpa armigera stunt virus, FAW-macule-like virus or Spodoptera frugiperda rhabdovirus.

26. The expression construct of claim 17, wherein the nucleotide sequence that encodes a transport peptide encodes a protein having between 70% and 100% sequence identity to any one of SEQ ID Nos: 1-4.

27. The expression construct of claim 19, wherein the plant pathogen is selected from the group consisting of lepidopteran, coleopteran, dipteran, hemipteran, and nematode pest.

28. The expression construct of claim 27, wherein the plant pathogen is CEW, SAW, FAW, Helicoverpa armigera or Stink bug.

29. The expression construct of claim 19, wherein the expressed transport protein operably fused to the cargo molecule enters the hemocoel of an insect.

30. The expression construct of claim 29, wherein the cargo molecule kills the insect or decreases the insect's growth.

31. A cell comprising the expression construct of claim 17.

32. The cell of claim 31, wherein the cell is a bacterial cell, a plant cell, a yeast cell or an insect cell.

33.-58. (canceled)
